(19) United States
(12) Patent Application Publication
(10) Pub. No.: US 2017/0241974 A1
(43) Pub. Date: Aug. 24, 2017

(54) DETERMINATION OF FAME CONTENT IN FUEL

(71) Applicants: Pauline William ZARD, London (GB); PALOX OFFSHORE S.A.L., Jal El Did (LB)

(72) Inventor: William A. ZARD, Jal el Dib (LB)

(73) Assignee: Pauline William ZARD, Jal El Did (LB)

(21) Appl. No.: 15/504,745

(22) PCT Filed: Aug. 19, 2015
(86) PCT No.: PCT/EP2015/069031

§ 371 Date: Feb. 17, 2017

(30) Foreign Application Priority Data
Aug. 19, 2014 (GB) 1414686.4

(51) Int. Cl. G01N 33/28; C10L 1/02; G01N 21/78

(52) U.S. Cl. CPC G01N 33/2835; G01N 21/78; C10L 1/02; C10L 2290/60

(57) ABSTRACT

A method for determining whether a liquid hydrocarbon fuel, contained in a reservoir, comprises no more than a specified maximum permissible amount of fatty acid methyl ester (FAME) for the fuel to be accepted as being suitable for use in a specified engine.

### FIELD OF THE INVENTION

This invention is concerned with a method for determining whether a liquid hydrocarbon fuel is suitable for use in a particular engine. More particularly, the invention concerns a method for determining whether a liquid hydrocarbon fuel comprises an acceptable amount or concentration of fatty acid methyl ester (FAME).

### BACKGROUND

FAME is now a common bio-derived component in middle distillate fuels. With many different natural oil sources available (e.g. palm oil, rape seed oil, etc), the detailed composition can be variable in terms of the fatty acid component, but the alcohol used to make the ester from the natural oil is always methanol, hence the “methyl ester” part of the name. Typically, the formation of FAME can be represented by the following reaction scheme:

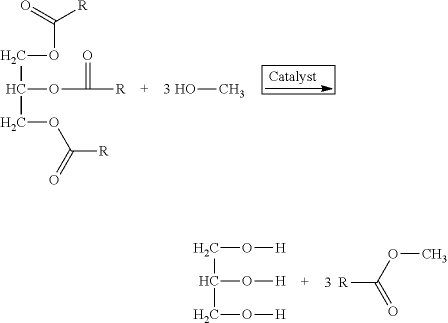

where R is a fatty alkyl group. FAME is the R—(C═O)—OCH₃ reaction product.

The amount or concentration of FAME that can be suitably employed in a liquid hydrocarbon fuel depends upon the type of fuel and the engine for which the fuel is destined for use.

Legislation in various countries relevant to the automotive industry permits FAME to be used in certain liquid hydrocarbon fuels in significant amounts (percentage levels). For example, currently in the European Union (EU), legislation permits FAME to be added to diesel fuel in an amount up to a maximum of 8 percent by weight when the FAME-containing fuel is destined for sale at retail filling stations for general commercial automotive vehicles. Higher percentage FAME-containing diesel fuels are available, but these fuels are destined for use in vehicles having specially adapted engines.

In the aviation industry, however, FAME in jet fuel is considered a contaminant, with a “detectable limit” of 5 ppm (mg/Kg) FAME being the current maximum permitted amount. The implication is that if you can detect FAME then the jet fuel is not fit for purpose. Recent work by the aviation industry has suggested that the maximum limit may be relaxed up to 100 ppm FAME, but it is likely that the limit will be raised cautiously to 50 ppm in the first instance so that any unforeseen issues can be detected without safety implications, though these new limits have yet to be adopted by the industry stakeholders.

Liquid hydrocarbon fuels are often transported to storage terminals and fuel farms and then distributed to commercial consumers, e.g. power stations and airports, and wholesale distribution depots, e.g. tanker stations, through a network of product pipelines. For logistical and economic reasons, at least some of the pipelines in the network of pipelines through which the fuel is pumped will be used to carry other fuel products i.e. multiproduct pipelines. Having no physical barrier between fuel products in the pipelines means that some interface mixing of fuel products will occur between different products. Cross-product contamination therefore becomes a concern, although pipeline management strategies are employed to try to minimize this. Nevertheless, trace levels (parts per million) of FAME from diesel products are known to contaminate other fuel products, such as jet fuel, that are transported in the pipeline. Indeed, various crises in the supply chains for jet fuel have only narrowly been averted, but in some isolated cases airports have been closed down because the FAME limit in the jet fuel has been exceeded.

One issue that challenges the fuel producers, distributors and aviation industry is the actual detection of FAME in jet fuel. The “detectable limit” of 5 ppm is currently only measurable accurately using sophisticated laboratory-based instruments, such as Gas Chromatography-Mass Spectrometry (GCMS), which are expensive and can lead to significant delays in confirming results to operators in the field. The potential increase on the limit to 50 ppm or 100 ppm offers to alleviate the problems to some extent, as less accurate but more portable field based screening instruments can be used.

United Kingdom patent application GB-A-2466802 (Stanhope-Seta Ltd) discloses an apparatus for measuring FAME content in jet fuel at levels down to 30 ppm that may be used at many points in the supply chain. The apparatus employs a method in which Fourier Transform Infra-red spectroscopy (FTIR) is used to provide a chemical fingerprint of a sample being analysed and the amount of FAME determined therefrom. According to promotional literature available from Seta Analytics (www.seta-analytics.com, FIJI FAME in Jet Instrument), a field operator will take about 20 minutes to complete analysis of the sample, and so determine the suitability of the fuel from which the sample is extracted.

United States patent application US 2012/0052857 (Eldridge et al) discloses a colorimetric method for detecting biodiesel in fuel. The method involves adding hydroxylamine and sodium hydroxide to a sample of fuel, heating the sample and then adding hydrochloric acid and iron (III) chloride with the appearance of a violet or pink colour indicating that biodiesel is present.

The object of the present invention is to provide an alternative method that does not use GCMS and/or FTIR for determining whether a liquid hydrocarbon fuel is suitable for use in a particular engine, with a preference that the method can be run in an apparatus that can be operated by a field technician and is cheaper to produce than GCMS or FTIR apparatus. Alternatively, or preferably in addition, an object of the present invention is to provide a method for determining whether a liquid hydrocarbon fuel is suitable for use in a particular engine that may be carried out faster than the current methods that use GCMS and FTIR, with a preference that the method can be run in an apparatus that can be operated by a field technician and is cheaper to produce than GCMS or FTIR apparatus.

### STATEMENT OF THE INVENTION

The present invention provides a method for determining whether a liquid hydrocarbon fuel, contained in a reservoir, comprises no more than a specified maximum permissible amount of fatty acid methyl ester (FAME) for the fuel to be accepted as being suitable for use in a specified engine, said method comprising the following sequential steps:
a) extracting a test sample of fuel from said reservoir;
b) contacting said test sample with water and a compound under reaction conditions that FAME, if present in said test sample, will undergo a de-esterification reaction to produce fatty acid and methanol in equimolar amounts, wherein the amounts of said water and said compound that are contacted with said test sample provide concentrations of said water and said compound in said test sample that are greater than the theoretical concentrations required to cause de-esterification of all FAME in an analogous control sample of a standard of the liquid hydrocarbon fuel which comprises said specified maximum permissible amount of FAME; and
c) determining whether the amount of fatty acid and/or methanol in said test sample after step b) is more than the amount of fatty acid and/or methanol obtained when said analogous control sample is subjected to identical de-esterification reaction conditions but using said theoretical concentrations of said water and said compound;

wherein the fuel in the reservoir is either:
i) accepted as being suitable for use in a specified engine when the amount of fatty acid and/or methanol in said test sample determined in step c) is no more than the amount obtained in said analogous control sample; or
ii) considered unsuitable for use in a specified engine when the amount of fatty acid and/or methanol in said test sample determined in step c) is greater than the amount obtained in said analogous control sample.

Preferably in step c) the amount of fatty acid and methanol in said test sample is determined.

Preferably, the amount of said water and said compound that is contacted with the test sample provides a concentration of said water and said compound in said test sample that is greater than that concentration required to cause de-esterification of all FAME in the test sample prior to step b).

Preferably, said water and said compound are contacted with said liquid hydrocarbon fuel by mixing them all together in a vessel and exposing the mixture to agitation conditions, such as by a mechanical mixer and/or by shaking.

### TERMS

The term “fatty acid methyl ester” or “FAME” as used herein means a fatty acid ester or a mixture of fatty acid esters that are derived by transesterification of fats with methanol.

The term “fuel” as used herein means a liquid hydrocarbon or mixture of hydrocarbons that is suitable for use as fuel for an engine. Preferably the fuel jet fuel or diesel, but especially jet fuel.

Throughout this specification and in the claims that follow, unless the context requires otherwise, the word “comprise” or variations such as “comprises” and “comprising”, will be understood to imply the inclusion of a stated integer or group of integers but not the exclusion of any other stated integer or group of integers.

### DETAILED DESCRIPTION

This present invention is a method for determining whether a liquid hydrocarbon fuel is suitable for use in a particular engine by determining whether it comprises an acceptable amount of fatty acid methyl ester (FAME).

The de-esterification reaction that occurs in step c), if FAME is present in the sample, can be represented by the following reaction scheme:

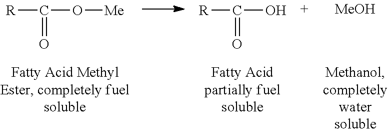

where R is a fatty alkyl group and Me is a methyl group.

The liquid hydrocarbon fuel is preferably jet fuel or diesel, more preferably jet fuel.

The method can be carried out under aqueous conditions or non-aqueous conditions as desired.

Preferably, the compound that, with the water, is contacted with the sample of liquid hydrocarbon fuel is selected from one or more enzymes, preferably an esterase, peroxidase, lipase, phosphoesterase and/or hydrolase, and mixtures thereof; more preferably an EC1 oxidoreductase and/or EC3 hydrolase. The performance of the enzyme preferably enhanced by the use of a cofactor, such as one or more weak acids (i.e. a weak acid is an acid that dissociates incompletely, releasing only some of its hydrogen atoms into the solution. Thus, it is less capable than a strong acid of donating protons) e.g. boric acid and/or citric acid; one or more peroxides, such as H₂O₂; and mixtures thereof. Strong acids are not preferred due to safety concerns.

A person skilled in the art will recognise that the de-esterification reaction is an equilibrium reaction and that, accordingly, the yield of the fatty acid and the methanol will be determined by the particular reaction conditions (i.e. temperature, time and pressure) to which the reaction components are exposed. For example, a person skilled in the art will recognise that if 50 moles of FAME are subjected to reaction conditions wherein only 80% of the FAME undergoes de-esterification then, after de-esterification, 40 moles of fatty acid and 40 moles of methanol will be formed.

FAME is completely soluble in the liquid hydrocarbon fuel, whereas the fatty acid(s) produced by the de-esterification reaction may be only partially soluble in the fuel. The water that is contacted with the liquid hydrocarbon fuel in step b) is completely immiscible with the fuel, and so will form a separate phase to the fuel when the sample is left to stand for a short period of time (e.g. at most only a few seconds). The methanol produced in the de-esterification reaction is completely soluble in the water.

As the methanol is also soluble in the fuel some of the methods used to determine its concentration, and by inference the concentration of FAME, can be performed without the need for extraction into an aqueous phase. Once such method is that using the colorimeter.

Whilst it is possible to analyse and determine the amount of fatty acid formed in the sample, e.g. through the use of suitable chromophores/fluorophores, because methanol is completely soluble in the water and analytical methods sensitive to ppm levels are well established for such aqueous systems, it is relatively easier for a person skilled in the art to determine whether the concentration of methanol in the water phase of the test sample after step b) is greater than the concentration of methanol that would have been formed in the water phase of the analogous control sample. Thus, it is preferable that step c) comprises determining whether the test sample after step b) contains methanol in an amount more than the amount of methanol obtained when said analogous control sample is subjected to identical de-esterification reaction conditions but using said theoretical concentrations of said water and said compound.

If the amount of fatty acid and/or methanol obtained from a test sample is greater than the amount of fatty acid and/or methanol obtained from the analogous control sample, it can be concluded that the amount of FAME in the test sample of fuel will be above the specified maximum permissible amount. It may therefore be concluded that the fuel in the reservoir from which the test sample was extracted will also contain FAME in an amount greater than the maximum permissible amount. The fuel in the reservoir should therefore be considered unsuitable for use in the specified engine.

If the amount of fatty acid and/or methanol obtained from a test sample is equal to or less than the amount of fatty acid and/or methanol obtained from the analogous control sample, it can be concluded that the amount of FAME in the test sample of fuel will be equal to or less than the specified maximum permissible amount. It may therefore be concluded that the fuel in the reservoir from which the test sample was extracted will not contain FAME in an amount greater than the maximum permissible amount. The fuel in the reservoir should therefore be considered as acceptable for use in the specified engine.

Determining whether the amount of fatty acid and/or methanol in said test sample after step b) is more than the amount of fatty acid and/or methanol obtained when said analogous control sample is subjected to identical de-esterification reaction conditions but using said theoretical concentrations of said water and said compound may be conducted qualitatively or quantitatively.

An analogous control sample is a sample taken from a standard liquid hydrocarbon fuel that comprises a known amount of FAME and which is tested under substantially identical testing conditions e.g. temperature, time and pressure as the test sample. The control sample can be used to calibrate any equipment used in the determination method of the present invention and/or it may be used to prepare a calibrated visual display which can be used for comparative purposes.

Various qualitative and quantitative methods for determining the amount of methanol in a test sample, and consequently the equivalent amount of FAME, are available to the skilled person. For example:

i) after contacting a test sample of fuel with sufficient water and compound and allowing the mixture to settle, a portion of the aqueous phase that is formed may be tested with an alcohol test strip which exhibits a particular colour when exposed to a specified amount of methanol or an alcohol test strip that exhibits a range of colours when exposed to various amounts of methanol can be used to provide a calibrated visual display wherein the particular colour exhibited equates to an amount of FAME in the fuel;

ii) a test sample of fuel may be contacted with sufficient water and compound in a colorimeter and the light absorbance measured. Comparison of the measured wavelength against a calibration standard graph will provide an indication of the amount of FAME in the fuel;

iii) after contacting a test sample of fuel with sufficient water and compound and allowing the mixture to settle, a portion of the aqueous phase that is formed may be tested with a modified breathalyser, where the calibrated display reading for an amount of methanol is equated to an amount of FAME in the sample.

Step c) preferably comprises extracting a portion of a water phase produced in the test sample during step b), and determining qualitatively or quantitatively the concentration of methanol in the extracted portion of the water phase by comparison to methanol concentrations obtained for one or more analogous control sample(s). In one embodiment, step c) comprises extracting a portion of a water phase produced in the test sample during step b), determining qualitatively the concentration of methanol in the extracted portion of the water phase by applying an amount of the water phase to an alcohol test strip that is colour sensitive to methanol, observing the colour displayed on the alcohol test strip and comparing that observed colour against a single colour wherein that colour is indicative of a colour obtained from a specific analogous control sample: this particular embodiment can be used to provide a yes/no determination as to whether the fuel in the reservoir contains more than the specified maximum permissible amount of FAME. In another embodiment step c) comprises extracting a portion of a water phase produced in the test sample during step b), determining qualitatively the concentration of methanol in the extracted portion of the water phase by applying an amount of the water phase to an alcohol test strip that is colour sensitive to methanol, observing the colour displayed on the alcohol test strip and comparing that observed colour against a plurality of colours on a chart wherein each colour on the chart is indicative of a colour obtained from a plurality of specific analogous control samples: whilst this particular embodiment can be used to provide a yes/no determination as to whether the fuel in the reservoir contains more than the specified maximum permissible amount of FAME, it may also be used to provide an approximation of the actual concentration of FAME in the fuel in the reservoir.

The present invention shall now be further illustrated by way of the following example.

The following abbreviations are used in the Examples:

DI Deionised

FAME Fatty acid methyl ester

GCMS Gas chromatography-mass spectrometry

RME Rapeseed methyl ester

### EXAMPLES

### Example 1

### Preparation of Control Sample

In an open laboratory held at about 20° C., a quantity of rapeseed methyl ester (RME) was added to 5 litres of Jet A-1 fuel in a portable hand-held fuel can, so as to provide a FAME concentration of 50 ppm (mg/Kg). 500 ml of the jet fuel comprising RME was extracted from the fuel can as a test sample and put into an open beaker. 50 ml of DI water, 505 mg of hydrolase and 505 mg of lipase were added to the beaker and the contents mixed vigorously. After about 10 seconds, the contents had settled, providing a clear water phase at the bottom of the beaker and a clear fuel phase on top of the water phase.

Immediately thereafter, using a pipette, 10 ml of the water phase is extracted from beneath the fuel phase and placed into a sealed first test tube.

A 1 ml portion of the extracted water phase is then extracted from the test tube and evaluated for methanol content using GCMS. It can be determined that the water phase contained methanol in a concentration of 104 ppm, which indicated that about 20% of the RME had been de-esterified in the equilibrium reaction.

### Example 2

### Preparation of Test Samples

Two differing amounts of RME were added to a second and a third portable hand-held fuel cans each containing 5 litres of Jet A-1 fuel, so as to provide a FAME concentration in the second can of 40 ppm and a FAME concentration in the third can of 60 ppm. 500 ml of the jet fuel comprising RME is extracted from each fuel can and put into two open beakers.

50 ml of DI water, 505 mg of hydrolase and 505 mg of lipase are then added to each beaker and the contents mixed vigorously for 3 minutes. After about 10 seconds, the contents settle out, so providing a clear water phase at the bottom of the beaker and a clear fuel phase on top of the water phase.

Immediately thereafter, using a pipette, 10 ml of the water phase from each beaker was extracted from beneath the fuel phase and placed into two sealed second and third test tubes.

### Example 3

### Determination of FAME Concentration Using a Breathalyser Device

A hand-held breathalyser, as may be used by police for taking breath tests from motorists suspected of driving with alcohol levels over the legal maximum, e.g. ALCOSENSE ZENITH+breathalyser (AlcoSense), is modified to receive vapourized contents of the first test tube at 37° C. for 5 seconds, and the breathalyser adjusted to calibrate it to indicate 100 on the visual display.

Vapourized contents of the second test tube at 37° C. are then passed in to the breathalyser for 5 seconds and the display observed. The display indicates a reading less than 100.

Vapourized contents of the third test tube at 37° C. are then passed in to the breathalyser for 5 seconds and the display observed. The display indicates a reading more than 100.

From the above, it can be determined that the second fuel can contains less RME than the analogous control sample and the third fuel can contains more RME than the analogous control.

If 50 ppm FAME was the specified maximum permissible amount for a jet fuel to be suitable for use in a Rolls Royce Trent jet engine, then the fuel in the second fuel can would be accepted, whereas the fuel in the third fuel can would be considered unacceptable and so rejected.

Direct methanol measurements can be made using analytical alcohol detection equipment such as the ANALOX AM5 analyser (Analox Instruments). These results are given as a quantitative value expressed as parts per million.

### Example 4

### Determination of FAME Concentration Using an Alcohol Test Strip

The following test was performed using a ZAP methanol test kit from Clinitox Diagnostix.

A drop of water phase extracted from each of the first, second and third test tubes was applied to three identical alcohol test strips that exhibit different colours depending upon the amount of methanol that it is exposed to. After about two minutes, each test strips was observed to show a different colour to the other two test strips. The colour of the test strip associated with the second test tube indicated less methanol than the colour of the test strip of the first test tube, whereas the colour of the test strip associated with the third test tubes indicated more methanol than the colour of the test strip of the first test tube.

Assuming 50 ppm FAME was the specified maximum permissible amount for a jet fuel to be suitable for use in a jet engine, the results of these test strips indicated that the fuel in the second fuel can would be accepted, whereas the fuel in the third fuel can would be considered unacceptable and so rejected.

If the colour of the test strip associated with the first test tube is accepted as being the colour of a strip for a standard fuel comprising 50 ppm maximum FAME, then that can be accepted as the calibration standard colour against which other samples may be tested and qualitatively determined for their suitability, provided that they are subjected to an analogous test procedure.

### Example 5

### Preparation of Test Fluid Containing Esterase and Hydroperoxy Fatty Reductase

A test fluid was prepared by mixing 100 g of each of esterase (Sigma) and hydroperoxy fatty reductase (Sigma), dissolving the mixture in odourless kerosene and making the test fluid up to 1 litre in volume.

### Example 6

### Preparation of Test Fluid Containing 2,4,6-dichloro-1,3,5-triazine

A test fluid was prepared by dissolving 2.5 g of 2,4,6-dichloro-1,3,5-triazine in odourless kerosene (which is similar chemically to jet fuel) and making the test fluid up to 1 litre in volume.

### Example 7

### Determination of FAME Concentration Using a Colorimeter

A series of jet fuels were prepared containing known amounts of fatty acid methyl ester derived from both rapeseed and soy (i.e. some samples contained rapeseed-derived FAME and some samples contained soy-derived FAME). These fluids (500 ml) were treated with 10 ml of the fluid from Example 5 and shaken for 2 minutes. After this time 10 ml of the test fluid from Example 6 was added and shaken for 30 seconds. A reading was immediately taken in a colorimeter (Vernier N450) at a given wavelength (this can be varied to optimize the sensitivity of the test but typically is either 565 nm or 635 nm) followed by a second reading in the same colorimeter 1 minute later. The difference between these readings is directly related to the amount of FAME present. A calibration graph was produced from these results.

The results are given in Table 1 below:

TABLE 1

| | FAME | Initial Reading at | Final Reading at | Change in |
|---|---|---|---|---|
| FAME Type | Level | 635 nm | 635 nm | Transmission |
| Rapeseed | 0 | 80.1 | 80.2 | 0.1 |
| Rapeseed | 5 | 81.2 | 83.6 | 2.4 |
| Rapeseed | 40 | 81.9 | 87.8 | 5.9 |
| Rapeseed | 50 | 82.1 | 88.6 | 6.5 |
| Rapeseed | 100 | 82.5 | 89.6 | 7.1 |
| Soy | 0 | 79.8 | 79.8 | 0 |
| Soy | 5 | 80.1 | 82.3 | 2.2 |
| Soy | 40 | 81.6 | 87.2 | 5.7 |
| Soy | 50 | 82.2 | 88.5 | 6.3 |
| Soy | 100 | 82.5 | 89.5 | 7.0 |

The results show a direct relationship between the change in Transmission with the amount of FAME present.

### Example 8

### Determination of FAME Concentration Using a Colorimeter

A jet fuel containing an unknown amount of FAME was treated in exactly the same manner as the jet fuels were treated in Example 7. The difference between initial and final colorimeter readings was read on the calibration graph to determine an approximate level of FAME in the fluid. The FAME concentration of the jet fuel was determined to be 30 ppm that was later verified using GCMS techniques.

This demonstrated that the method can determine the concentration of FAME in samples of jet fuel.

### Example 9

### Preparation of Test Fluid Containing an Esterase, a Lipase, a Fatty Acid Peroxidase and a Hydroperoxy Fatty Reductase

A test fluid was prepared by mixing 50 g of each of an esterase (Sigma), a lipase (Sigma), a fatty acid peroxidase (Sigma) and hydroperoxy fatty reductase (Sigma), dissolving the mixture in odourless kerosene and making the test fluid up to 1 litre in volume.

### Example 10

### Determination of FAME Concentration Using a Colorimeter

A series of Jet fuels were prepared containing known amounts of fatty acid methyl ester derived from both rapeseed and soy (i.e. some samples contained rapeseed-derived FAME and some samples contained soy-derived FAME). These fluids (500 ml) were treated with 10 ml of the fluid from Example 9 and shaken for 2 minutes. After this time 10 ml of the fluid from Example 6 was added and shaken for 30 seconds. A reading was immediately taken in a colorimeter (Vernier N450) at a given wavelength (this can be varied to optimize the sensitivity of the test but typically is either 565 nm or 635 nm) followed by a second reading one minute later. The difference between these readings is directly related to the amount of FAME present. A calibration graph was produced from these results.

The results are given in Table 2 below:

TABLE 2

| | FAME | Initial Reading at | Final Reading at | Change in |
|---|---|---|---|---|
| FAME Type | Level | 635 nm | 635 nm | Transmission |
| Rapeseed | 0 | 80.1 | 80.1 | 0 |
| Rapeseed | 5 | 81.2 | 83.8 | 2.6 |
| Rapeseed | 40 | 81.9 | 88.0 | 6.1 |
| Rapeseed | 50 | 82.1 | 89.0 | 6.9 |
| Rapeseed | 100 | 82.5 | 89.9 | 7.4 |
| Soy | 0 | 85.3 | 85.4 | 0.1 |
| Soy | 5 | 86.5 | 89.1 | 2.6 |
| Soy | 40 | 87.1 | 93.3 | 6.2 |
| Soy | 50 | 87.4 | 94.1 | 6.7 |
| Soy | 100 | 87.8 | 95.3 | 7.5 |

The results show a direct relationship between the change in Transmission with the amount of FAME present.

### Example 11

### Determination of FAME Concentration Using a Colorimeter

A jet fuel containing an unknown amount of FAME was treated in exactly the same manner as the jet fuels were treated in Example 10. The difference between initial and final colorimeter readings was read on the calibration graph to determine an approximate level of FAME in the fluid. FAME levels were confirmed by GCMS techniques.

This demonstrated that the method can determine the concentration of FAME in samples of jet fuel.

### Example 12

### Preparation of Test Fluid Containing a Mixture of a Lipase and a Fatty Acid Peroxidase

A test fluid was prepared by mixing 50 g of each of a lipase (Sigma), and a fatty acid peroxidase (Sigma), dissolving the mixture in odourless kerosene and making the test fluid up to 1 litre in volume.

### Example 13

### Determination of FAME Concentration Using a Colorimeter

A series of Jet fuels were prepared containing known amounts of fatty acid methyl ester derived from rapeseed. These fluids (500 ml) were treated with 10 ml of the fluid from Example 12 and shaken for 2 minutes. After this time 10 ml of the fluid from Example 6 was added and shaken for 30 seconds. A reading was immediately taken in a colorimeter (Vernier N450) at a given wavelength (this can be varied to optimize the sensitivity of the test but typically is either 565nm or 635nm) followed by a second reading 1 minute later. The difference between these readings is directly related to the amount of FAME present. A calibration graph was produced from these results. The results are given in Table 3 below:

TABLE 3

| | FAME | Initial Reading at | Final Reading at | Change in |
|---|---|---|---|---|
| FAME Type | Level | 635 nm | 635 nm | Transmission |
| Rapeseed | 0 | 80.4 | 80.4 | 0 |
| Rapeseed | 5 | 81.0 | 83.4 | 2.4 |
| Rapeseed | 40 | 81.7 | 87.2 | 5.5 |
| Rapeseed | 50 | 81.9 | 89.0 | 7.1 |
| Rapeseed | 100 | 82.4 | 90.1 | 7.7 |

This test was only carried out with rapeseed methyl ester.

The results show a direct relationship between the change in Transmission with the amount of FAME present.

### Example 14

### Determination of FAME Concentration Using a Colorimeter

A jet fuel containing an unknown amount of FAME was treated in exactly the same manner as those in Example 13. The difference between initial and final colorimeter readings were read on the calibration graph to determine an approximate level of FAME in the fluid. FAME levels were confirmed by GCMS techniques.

This demonstrated that the method can determine the concentration of FAME in samples of jet fuel.

1. A method for determining whether a liquid hydrocarbon fuel, contained in a reservoir, comprises no more than a specified maximum permissible amount of fatty acid methyl ester (FAME) for the fuel to be accepted as being suitable for use in a specified engine, said method comprising the following sequential steps:
a) extracting a test sample of fuel from said reservoir;
b) contacting said test sample with water and a compound under reaction conditions that FAME, if present in said test sample, will undergo a de-esterification reaction to produce a fatty acid and methanol in equimolar amounts, wherein the amounts of said water and said compound that are contacted with said test sample provide concentrations of said water and said compound in said test sample that are greater than the theoretical concentrations required to cause de-esterification of all FAME in an analogous control sample of a standard of the liquid hydrocarbon fuel which comprises said specified maximum permissible amount of FAME; and
c) determining whether the amount of methanol in said test sample after step b) is more than the amount of methanol obtained when said analogous control sample is subjected to identical de-esterification reaction conditions but using said theoretical concentrations of said water and said compound;
wherein the fuel in the reservoir is either:
i) accepted as being suitable for use in a specified engine when the amount of methanol in said test sample determined in step c) is no more than the amount obtained in said analogous control sample; or
ii) considered unsuitable for use in a specified engine when the amount of methanol in said test sample determined in step c) is greater than the amount obtained in said analogous control sample.

2. A method as claimed in claim 1, wherein step c) comprises determining whether the amount of fatty acid and methanol in said test sample after step b) is more than the amount of fatty acid and methanol obtained when said analogous control sample is subjected to identical de-esterification reaction conditions, and wherein the fuel in the reservoir is either:
i) accepted as being suitable for use in a specified engine when the amount of fatty acid and methanol in said test sample determined in step c) is no more than the amount obtained in said analogous control sample; or
ii) considered unsuitable for use in a specified engine when the amount of fatty acid and methanol in said test sample determined in step c) is greater than the amount obtained in said analogous control sample.

3. A method as claimed in claim 1, wherein the amount of said compound that is contacted with said test sample provides a concentration of said compound in said test sample that is greater than that concentration required to cause de-esterification of all FAME in the test sample prior to step b).

4. A method as claimed in claim 1, wherein step c) comprises determining whether the test sample after step b) contains methanol in an amount more than the amount equivalent to said specified maximum permissible amount of FAME.

5. A method as claimed in claim 1, wherein step c) comprises extracting a portion of a water phase produced in the test sample during step b), determining qualitatively or quantitatively the concentration of methanol in the extracted portion of the water phase by comparison to methanol concentrations obtained for one or more analogous control sample(s).

6. A method as claimed in claim 5, wherein step c) comprises extracting a portion of a water phase produced in the test sample during step b), determining qualitatively the concentration of methanol in the extracted portion of the water phase by applying an amount of the water phase to an alcohol test strip that is colour sensitive to methanol, observing the colour displayed on the alcohol test strip and comparing that observed colour against a single colour wherein that colour is indicative of a colour obtained from a specific analogous control sample.

7. A method as claimed in claim 5, wherein step c) comprises extracting a portion of a water phase produced in the test sample during step b), determining qualitatively the concentration of methanol in the extracted portion of the water phase by applying an amount of the water phase to an alcohol test strip that is colour sensitive to methanol, observing the colour displayed on the alcohol test strip and comparing that observed colour against a plurality of colours wherein each colour is indicative of a colour obtained from a plurality of specific analogous control samples.
